SEQUENCE LISTING (3 sequences)

SEQ ID NO: 1 - protein, 431 aa, unidentified
  source 1..431: note = Lasiodiplodia iraniensis
MSKPTQQLGQPLSFPISGRTAPNRFLKGAMSERLASWDPHPLSRGVPSRELAELYEVWGRGQIGVLVTGNIMVDPAHPEAVGNAAIPFGASESSRRFEAFRDMARAGKKHGSLMIAQLSHPGRQCDVRIQPDPVSASDVQLQGTVMGMRFGKPYAAGREEISRIVRQFQFAATYLEQAGFDGVEVHAAHGYLVAQFLSPTTNKRGDEYGGGSLEGRARLLVEIVRAIRAGTSPGFVVGVKLNSVEFQQDGLRPEEARELCAVLDGMGVDFVELSGGTYEQLAFKHERESSRKREAFFIEFAALIAPELRRAKTYLTGGFQTAAGMADALATVDGVGLGRALCHEPFLCRDILAGSTAGVARSLVDGNNAALSNAAALMQMWRLALGKEPWDTSTPEGMSAMLQLLARYRKEFERADFTTPGDFRRTILAVL

SEQ ID NO: 2 - DNA, 1296 bp, unidentified
  source 1..1296: mol_type = genomic DNA; note = Lasiodiplodia iraniensis
atgtccaaacccactcaacaactcggccagcccctttcatttcccatctccggcaggactgcccccaaccggttcctcaaaggagccatgtcggaacgtcttgcctcctgggatccgcatcccctcagccgcggggtgccttcgcgggagctggcggagctttacgaggtctggggacgtggccagattggcgtcctcgtgactggaaacatcatggtcgaccccgcgcacccggaggccgtgggcaacgcggctatcccttttggtgcgtcggaatcatcccgccggttcgaagcgttcagggacatggcacgcgcgggaaaaaagcacggaagcctgatgattgcccaactatctcatccgggccgtcagtgcgacgtgcggatccagcctgacccagtgtctgcctcggacgtccagctccaggggacagtgatggggatgcgtttcggcaagccatacgccgcaggccgcgaagaaatttcgaggatcgtccgacagttccaattcgcggccacgtacctcgagcaggctggattcgacggcgtcgaggtgcacgcggcccacggctacctcgtcgcgcagttcctatccccgacgaccaacaagcgcggcgacgagtacggcggcggcagcctcgagggccgcgcccggctgctcgtcgagatcgtgcgtgccatccgcgccggcacctctcccggcttcgtggtcggcgtcaagctcaacagcgttgagttccagcaggacgggctgcggcccgaggaggcgcgcgagctgtgcgccgtgctggacggcatgggcgtcgactttgtcgagctgtccggcggcacctacgagcagctcgccttcaaacacgagagggaatcctcgaggaagcgcgaggccttcttcatcgagtttgctgcgctgattgcgccggagttgcggagggcaaagacgtacctcactggcggcttccagacggctgctggcatggcggatgcgctggcgactgtcgatggcgtggggctcggccgcgccctgtgccatgagccgtttctctgtcgtgacatcttggctgggtcgacggctggagtggcgaggagcctcgttgatgggaataatgcagcgttgagtaacgccgctgccctgatgcaaatgtggagactcgcgctgggcaaggagccgtgggatacctctaccccggaaggtatgagtgctatgctccagctccttgctcgctaccggaaagagttcgagcgggcagattttactacccccggcgactttcgaagaacaattcttgcagtgctatag

SEQ ID NO: 3 - DNA, 1296 bp, unidentified
  source 1..1296: mol_type = other DNA; note = Lasiodiplodia iraniensis
atgagtaagccgacccagcagctgggtcagccgctgagcttcccgattagcggtcgtaccgcaccgaatcgcttcctgaaaggtgccatgagcgaacgtctggccagctgggatccgcatccgctgagccgtggtgttccgagccgtgaactggccgaactgtatgaagtgtggggccgtggccagattggcgttctggtgaccggcaatattatggttgatccggcccatccggaagccgttggtaatgccgccattccgttcggtgcaagtgaaagtagccgtcgcttcgaagcattccgtgatatggcccgcgcaggcaaaaaacatggtagtctgatgattgcccagctgagccatccgggccgtcagtgtgatgtgcgcattcagccggatccggtgagtgccagcgatgttcagctgcagggtaccgtgatgggtatgcgcttcggtaaaccgtatgccgccggtcgtgaagaaattagccgtattgttcgccagttccagttcgccgccacctatctggaacaggcaggcttcgatggtgtggaagtgcatgccgcccatggctatctggttgcccagttcctgagtccgaccaccaataaacgtggtgatgaatatggtggtggtagcctggaaggtcgtgcccgtctgctggttgaaattgttcgtgccattcgtgccggcaccagcccgggcttcgttgtgggtgtgaaactgaatagtgttgaattccagcaggatggcctgcgtccggaagaagcacgcgaactgtgtgcagttctggatggtatgggtgttgacttcgtggaactgagtggcggtacctatgaacagctggcattcaaacatgaacgcgaaagcagccgtaaacgtgaagcattcttcattgaattcgcagccctgattgcaccggaactgcgtcgcgccaaaacctatctgaccggcggcttccagaccgccgccggtatggctgatgcactggccaccgttgatggcgttggtctgggtcgcgccctgtgccatgaaccgttcctgtgccgcgatattctggcaggcagcaccgcaggcgttgcccgtagtctggtggatggtaataatgccgccctgagcaatgcagcagccctgatgcagatgtggcgtctggcactgggtaaagaaccgtgggataccagtaccccggaaggtatgagcgccatgctgcagctgctggcccgttatcgtaaagaattcgaacgcgccgacttcaccaccccgggtgacttccgtcgcaccattctggcagttctgtaa

(19) United States
(12) Patent Application Publication
(10) Pub. No.: US 2025/0137017 A1
(43) Pub. Date: May 1, 2025

(54) METABOLICALLY ENGINEERED CELLS FOR JASMONIC ACID BIOSYNTHESIS

(71) Applicant: Sumitomo Chemical Company, Limited, Tokyo (JP)

(72) Inventors: David Nunn, Lowell, MA (US); Hui Chen, North Billerica, MA (US)

(73) Assignee: Sumitomo Chemical Company, Limited, Tokyo (JP)

(21) Appl. No.: 18/915,912

(22) Filed: Oct. 15, 2024

Related U.S. Application Data
(63) Continuation of application No. PCT/JP2023/014273, filed on Apr. 6, 2023.
(60) Provisional application No. 63/331,266, filed on Apr. 15, 2022.

(51) Int. Cl. C12P 7/40; C12N 1/16; C12N 1/20; C12N 5/04; C12N 9/02; C12R 1/645

(52) U.S. Cl. CPC C12P 7/40; C12N 1/16; C12N 1/20; C12N 5/04; C12N 9/001; C12Y 103/01042; C12N 2510/02; C12R 2001/645

(57) ABSTRACT

The present invention relates, in some aspects, to a host cell comprising a metabolic pathway producing jasmonic acid. The host cell overexpresses a polypeptide having 12-oxophytodienoate reductase (OPR) activity relative to a corresponding parental host cell.

### CROSS-REFERENCE TO RELATED APPLICATIONS

This Application is a Continuation of International Patent Application Serial No. PCT/JP2023/014273, filed Apr. 6, 2023, which claims the benefit of priority under 35 U.S.C. § 119(e) of U.S. Provisional Application No. 63/331,266, filed Apr. 15, 2022, both entitled “METABOLICALLY ENGINEERED CELLS FOR JASMONIC ACID BIOSYNTHESIS”. The entire contents of each of these applications are incorporated herein by reference.

### REFERENCE TO AN ELECTRONIC SEQUENCE LISTING

The contents of the electronic sequence listing (C149770083US01-JAV-SEQ.xml; Size: 7,228 bytes; and Date of Creation: Oct. 15, 2024) is herein incorporated by reference in its entirety.

### TECHNICAL FIELD

The field of the invention relates to cells, enzymes, and processes useful in the production of jasmonic acid molecules. More specifically, the present disclosure relates to a recombinant host cell overexpressing a polypeptide having 12-oxophytodienoate reductase (OPR) activity relative to a corresponding parental host cell.

### BACKGROUND ART

Production of chemicals from cell cultures is an important application of biotechnology. Typically, the steps in developing such a bio-production method may include (1) selection of a proper host cell, (2) elimination of metabolic pathways leading to undesirable by-products, (3) deregulation of desired pathways at both enzyme activity level and the transcriptional level, and (4) overexpression of appropriate enzymes in the desired pathways.

Jasmonic acid (“JA”) is an organic compound with a chemical formula of C₆H₆O₂. It and its derivatives are usually known as “jasmonates” and find a great number of applications in the agricultural, flavors and fragrances, and biopharmaceutical arts. Traditionally, filamentous fungi have been the only commercially viable source of biosynthetic jasmonates. However, the yields tend to be highly correlated with fungal morphology which raises a substantial challenge for the production of jasmonic acid in conventional fermenter apparatuses.

Higher plants are believed to synthesize jasmonic acid according to the biosynthetic pathway outlined in FIG. 4. The enzyme 12-Oxophytodienoate reductase (OPR, EC 1.3.1.42) catalyzes the reduction of 12-oxophytodienoic acid (12-OPDA) into 3-oxo-2-(cis-2′-pentenyl)cyclopentane-1-octanoic acid (OPC8), a key step in the biosynthetic pathway of jasmonic acid:

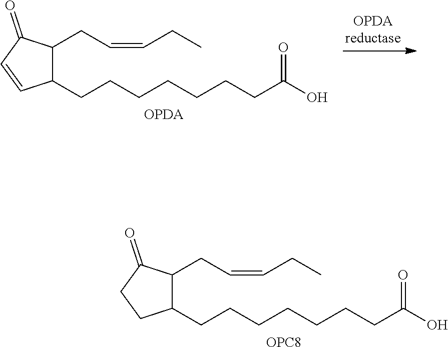

OPR belongs to the old yellow enzyme (OYE) family and utilizes NADPH and flavin mononucleotide (FMN) for reduction of the double bond of OPDA (Schaller and Weiler, 1997). Plant OPR enzymes dedicated to jasmonic acid biosynthesis have been well characterized and the corresponding genes have been cloned (Schaller and Weiler, 1997). In contrast, relatively little is known about fungal pathway of jasmonic acid biosynthesis despite the fact that jasmonic acid as a natural product was first isolated from Lasiodiplodia theobromae, a species of filamentous fungus (Aldridge et al., 1971).

Recent studies have shown that, similar to higher plants, fungi also use an allene oxide and OPDA as key intermediates for JA biosynthesis (Oliw and Hamberg, 2017), indicating that homology search may be used to identify fungal counterparts of plant genes related to the biosynthesis of jasmonic acid. However, it has been demonstrated that the cyclopentenone reduction mechanism in L. theobromae is different than that in plants. In plants, the reduced flavin cofactor appears to be placed on the α-face of the OPDA side chain whereas in known fungal enzyme, this occurs on the β-face of the cyclopentenone plane (Tsukada et al., 2010). Such differences in stereoselectivity of the cyclopentenone olefin reduction in plant and fungal OPR enzymes may suggest their differences in sequences and structures.

### CITATION LIST

### Non Patent Literature

[NPL1] Aldridge D C, Galt S, Giles D, and Turner W B. (1971) Metabolites of Lasiodiplodia theobromae. Journal of the Chemical Society C:1623-1627.
[NPL2] Ando A, Sumida Y, Ngoro H, Suroto D A. et al. (2009) Establishment of Agrobacterium tumefaciens-mediated transformation of an oleaginous fungus, Mortierella alpina 1S-4, and its application for eicosapentaenoic acid producer breeding. Appl. Environ. Microbiol. 75: 5529-5535.
[NPL3] de Groot M J, Bundock P, Hooykaas P J and Beijersbergen A G (1998). Agrobacterium tumefaciens-mediated transformation of filamentous fungi. Nat. Biotechnol. 16: 839-842.
[NPL4] dos Reis M C, Pelegrinelli Fungaro M H, Delgado Duarte R T, Furlaneto L, et al. (2004). Agrobacterium tumefaciens-mediated genetic transformation of the entomopathogenic fungus Beauveria bassiana. J. Microbiol. Methods 58: 197-202.
[NPL5] Hoekema A, Hirsch P R, Hooykaas P J J and Schilpercort R A (1983) A binary plant vector strategy based an separation of vir- and T-region of the Agrobacterium tumefaciens Ti-plasmid. Nature 303: 179-180.
[NPL6] Muniz C R, Freire F C O, Viana F M P, Cardoso J E, et al. (2012). Polyclonal antibody-based ELISA in combination with specific PCR amplification of internal transcribed spacer regions for the detection and quantitation of Lasiodiplodia theobromae, causal agent of gummosis in cashew nut plants. Ann. Appl. Biol. 160: 217-224,
[NPL7] Muniz C R, da Silva G F, Souza N I T, Freire F C O, Kema G H, and Guedes MIF (2014) Agrobacterium tumefaciens-mediated transformation of Lasiodiplodia theobroma, the causal agent of gummosis in cashew nut plants. Genet. Mol. Res. 13 (2): 2906-2913.
[NPL8] Oliw E H, Hamberg M. (2017) An allene oxide and 12-oxophytodienoic acid are key intermediates in jasmonic acid biosynthesis by Fusarium oxysporum. J Lipid Res. 58: 1670-1680.
[NPL9] Schaller F, Weiler E W. (1997) Molecular cloning and characterization of 12-oxophytodienoate reductase, an enzyme of the octadecanoid signaling pathway from Arabidopsis thaliana. Structural and functional relationship to yeast old yellow enzyme. J Biol Chem. 272: 2806-28072.
[NPL10] Sharma K K and Kuhad R C (2010). Genetic transformation of lignin degrading fungi facilitated by Agrobacterium tumefaciens. BMC Biotechnol. 10: 67.
[NPL11] Staats C C, Junges A, Fitarelli M, Furlaneto M C, et al. (2007). Gene inactivation mediated by Agrobacterium tumefaciens in the filamentous fungi Metarhizium anisopliae. Appl. Microbiol. Biotechnol. 76: 945-950.
[NPL12] Tsukada K, Takahashi K, Nabeta K. (2010) Biosynthesis of jasmonic acid in a plant pathogenic fungus, Lasiodiplodia theobromae. Phytochemistry, 71: 2019-2023.
[NPL13] Vieira A L and Camilo C M (2011) Agrobacterium tumefaciens-mediated transformation of the aquatic fungus Blastocladiella emersonii. Fungal. Genet. Biol. 48: 806-811.
[NPL14] Zheng P, Chen P C, Dong W H), 2019, Lasiodiplodia iranensis (A strain of Lasiodiplodia iranensis and its use for the production of jasmonic acid). China patent CN10722726413 tin Chinese).

### SUMMARY OF INVENTION

In a first aspect, the invention provides a recombinant host cell comprising a metabolic pathway producing jasmonic acid. The host cell overexpresses a polypeptide having 12-oxophytodienoate reductase (OPR) activity relative to a corresponding parental host cell. The polypeptide comprises an amino acid sequence having at least 90% identity to the amino acid sequence as set forth in SEQ ID NO:1.

In a second aspect, the invention encompasses a biosynthetic method for producing jasmonic acid, comprising: cultivating a recombinant host cell according to the aforesaid first aspect; and recovering the jasmonic acid from at least one of the recombinant cell and the culture medium.

In a third aspect, provided herein is a recombinant host cell comprising a metabolic pathway producing jasmonic acid, wherein the host cell overexpresses a gene encoding for a polypeptide having 12-oxophytodienoate reductase (OPR) activity relative to a corresponding parental host cell, wherein the gene comprises a polynucleotide sequence having at least 90% identity to the polynucleotide sequence as set forth in SEQ ID NO:2.

In a fourth aspect, provided herein is a biosynthetic method for producing jasmonic acid, the method comprising: cultivating the recombinant cell of the aforesaid third aspect; and recovering the jasmonic acid from at least one of the recombinant cell and the culture medium.

In a fifth aspect, provided herein is a biosynthetic method for producing a polypeptide having 12-oxophytodienoate reductase (OPR) activity, the method comprising: cultivating the recombinant cell of the aforesaid third aspect; and recovering the polypeptide having 12-oxophytodienoate reductase (OPR) activity from at least one of the recombinant cells and the culture medium.

While the disclosure is susceptible to various modifications and alternative forms, specific embodiments thereof are shown by way of example in the drawing and will herein be described in detail. It should be understood, however, that the drawings and detailed description presented herein are not intended to limit the disclosure to the particular embodiment disclosed, but on the contrary, the intention is to cover all modifications, equivalents, and alternatives falling within the spirit and scope of the present disclosure as defined by the appended claims.

Other features and advantages of this invention will become apparent in the following detailed description of preferred embodiments of this invention, taken with reference to the accompanying drawings.

### BRIEF DESCRIPTION OF DRAWINGS

FIG. 1A illustrates the purification of g5119 protein on an Ni-NTA column.

FIG. 1B illustrates the purification of g5119 protein on SDS-PAGE gel.

FIG. 2 includes a typical HPLC profile of a g5119-catalyzed reaction.

FIG. 3 includes LC/MS analyses of g5119-catalyzed reactions.

FIG. 4 illustrates the jasmonic acid biosynthetic pathway in higher plants.

FIG. 5 is a schematic representation of cassette hph-gs5119 cloning in the gateway destination binary vector pPm43GW.

### DESCRIPTION OF EMBODIMENTS

### Definitions

As used herein, the singular forms “a”, “an” and “the” include plural references unless the content clearly dictates otherwise.

To the extent that the term “include”, “have”, or the like is used in the description or the claims, such term is intended to be inclusive in a manner similar to the term “comprise” as “comprise” is interpreted when employed as a transitional word in a claim.

The word “exemplary” is used herein to mean “serving as an example, instance, or illustration”. Any embodiment described herein as “exemplary” is not necessarily to be construed as preferred or advantageous over other embodiments.

“Cellular system” is any cells that provide for the expression of ectopic proteins. It includes bacteria, yeast, filamentous fungi, plant cells and animal cells. It includes both prokaryotic and eukaryotic cells. It also includes the in vitro expression of proteins based on cellular components, such as ribosomes.

“Coding sequence” is to be given its ordinary and customary meaning to a person of ordinary skill in the art, and is used without limitation to refer to a DNA sequence that encodes for a specific amino acid sequence.

“Growing the Cellular System”. Growing includes providing an appropriate medium that would allow cells to multiply and divide. It also includes providing resources so that cells or cellular components can translate and make recombinant proteins.

“Protein Expression”. Protein production can occur after gene expression. It consists of the stages after DNA has been transcribed to messenger RNA (mRNA). The mRNA is then translated into polypeptide chains, which are ultimately folded into proteins. DNA may be present in the cells through transfection—a process of deliberately introducing nucleic acids into cells. The term is often used for non-viral methods in eukaryotic cells. It may also refer to other methods and cell types, although other terms are preferred: “transformation” is more often used to describe non-viral DNA transfer in bacteria, non-animal eukaryotic cells, including plant cells. In animal cells, transfection is the preferred term its transformation is also used to refer to progression to a cancerous state (carcinogenesis) in these cells. Transduction is often used to describe virus-mediated DNA transfer. Transformation, transduction, and viral infection are included under the definition of transfection for this application.

According to the current disclosure, a “filamentous fungus” or “mold” is a eukaryotic microorganism classified as a member of the fungus kingdom. Filamentous fungi typically grow in the form of multicellular filaments called hyphae. In contrast, fungi that can adopt a single-celled growth habit are called “yeasts”. Yeasts are unicellular organisms which evolved from multicellular ancestors. Yeast species useful for the current disclosure include but are not limited to those that have the ability to develop multicellular characteristics by forming strings of connected budding cells known as pseudohyphae or false hyphae.

The term “complementary” is to be given its ordinary and customary meaning to a person of ordinary skill in the art, and is used without limitation to describe the relationship between nucleotide bases that are, capable to hybridizing to one another. For example, with respect to DNA, adenosine is complementary to thymine and cytosine is complementary to guanine. Accordingly, the subject technology also includes isolated nucleic acid fragments that are complementary to the complete sequences as reported in the accompanying Sequence Listing as well as those substantially similar nucleic acid sequences.

The terms “nucleic acid” and “nucleotide” are to be given their respective ordinary and customary meanings to a person of ordinary skill in the art, and are used without limitation to refer to deoxyribonucleotides or ribonucleotides and polymers thereof in either single- or double-stranded form. Unless specifically limited, the term encompasses nucleic acids containing known analogues of natural nucleotides that have similar binding properties as the reference nucleic acid and are metabolized in a manner similar to naturally-occurring nucleotides.

As used herein, the term “nucleotide sequence” refers to a heteropolymer of nucleotides or the sequence of these nucleotides from the 5′ to 3′ end of a nucleic acid molecule and includes DNA or RNA molecules, including cDNA, a DNA fragment or portion, genomic DNA, synthetic, e.g. chemically synthesized, DNA, plasmid DNA. mRNA, and anti-sense RNA, any of which can be single stranded or double stranded. The terms “nucleotide sequence” “nucleic acid,” “nucleic acid molecule,” “oligonucleotide” and “polynucleotide” are also used interchangeably herein to refer to a heteropolymer of nucleotides. Nucleic acid molecules and/or nucleotide sequences provided herein are presented herein in the 5′ to 3′ direction, from left to right and are represented using the standard code for representing the nucleotide characters as set forth in the U.S. sequence rules, 37 CPR §§ 1.821-1.825 and the World Intellectual Property Organization (WIPO) Standard ST.25. Unless otherwise indicated, a particular nucleic acid sequence also implicitly encompasses conservatively modified or degenerate variants thereof (e.g., degenerate codon substitutions) and complementary sequences, as well as the sequence explicitly indicated.

As used herein, the term “gene” refers to at nucleic acid molecule capable of being used to produce mRNA, antisense RNA, miRNA, anti-microRNA antisense oligodeoxyribonucleotide (AMO), and the like. Genes may or may not be capable of being used to produce a functional protein or gene product. Genes can include both coding and non-coding regions, e.g. introns, regulatory elements, promoters, enhancers, termination sequences and/or 5′ and 3′ untranslated regions. A gene may be “isolated” by which is meant a nucleic acid that is substantially or essentially free from components normally found in association with the nucleic acid in its natural state. Such components include other cellular material, culture medium from recombinant production, and/or various chemicals used in chemically synthesizing the nucleic acid.

A “codon optimized” version of a gene refers to an exogenous gene introduced into a cell and where the codons of the gene have been optimized with regard to the particular cell. Typically, not all tRNAs are expressed equally or at the same level across species. Codon optimization of a gene sequence thereby involves changing codons to match the most prevalent tRNAs, i.e. to change a codon recognized by a low prevalent tRNA with a synonymous codon recognized by a tRNA that is comparatively more prevalent in the given fungal cell. This way the mRNA from the codon optimized gene will be more efficiently translated. The codon and the synonymous codon preferably encode the same amino acid.

“Genetical modification” or “genetically modified” as used herein involves such genetic modifications to the genome of the fungal cell, such as yeast, and/or introduction of exogenous nucleotide sequences, such as in the form of one or more plasmids, into the fungal cell, such as yeast.

The term “isolated” is to be given its ordinary and customary meaning to a person of ordinary skill in the art, and when used in the context of an isolated nucleic acid or an isolated polypeptidic, is used without limitation to refer to a nucleic acid or polypeptide that, by the hand of man, exists apart from its native environment and is therefore not a product of nature. An isolated nucleic acid or polypeptide can exist in a purified form or can exist in a non-native environment such as, for example, in a transgenic host cell.

The terms “incubating” and “incubation” as used herein means a process of mixing two or more chemical or biological entities (such as a chemical compound and an enzyme) and allowing them to interact under conditions favorable for producing a composition that includes a jasmonate compound.

The term “degenerate variant” refers to a nucleic acid sequence having a residue sequence that differs, from a reference nucleic acid sequence by one or more degenerate codon substitutions. Degenerate codon substitutions can be achieved by generating sequences in which the third position of one or more selected (or all) codons is substituted with mixed base and/or deoxy inosine residues. A nucleic acid sequence and all of its degenerate variants will express the same amino acid or polypeptide.

The terms “polypeptide,” “protein,” and “peptide” are to be given their respective ordinary and customary meanings to a person of ordinary skill in the art; the three terms are sometimes used interchangeably, and are used without limitation to refer to a polymer of amino acids, or amino acid analogs, regardless of its size or function. Although “protein” is often used in reference to relatively large polypeptides, end “peptide” is often used in reference to small polypeptides, usage of these terms in the art overlaps and varies. The term “polypeptide” as used herein refers to peptides, polypeptides, and proteins, unless otherwise noted. The terms “protein,” “polypeptide,” and “peptide” are used interchangeably herein when referring to a polynucleotide product. Thus, exemplary polypeptides include polynucleotide products, naturally occurring proteins, homologs, orthologs, paralogs, fragments and other equivalents, variants, and analogs of the foregoing.

The terms “polypeptide fragment” and “fragment,” when used in reference to a reference polypeptide, are to be given their ordinary and customary meanings to a person of ordinary skill in the art, and are used without limitation to refer to a polypeptide in which amino acid residues are deleted as compared to the reference polypeptide itself, but where the remaining amino acid sequence is usually identical to the corresponding positions in the reference polypeptide. Such deletions can occur at the amino-terminus or carboxy-terminus of the reference polypeptide, or alternatively both.

The term “functional fragment” of a polypeptide or protein refers to a peptide fragment that is a portion of the full-length polypeptide or protein, and has substantially the same biological activity, or carries out substantially the same function as the full-length polypeptide or protein (e.g., carrying out the same enzymatic reaction).

The terms “variant polypeptide,” “modified amino acid sequence” or “modified polypeptide,” which are used interchangeably, refer to an amino acid sequence that is different from the reference polypeptide by one or more amino acids, e.g., by ene or more amino acid substitutions, deletions, and/or additions. In an aspect, a variant is a “functional variant” which retains some or all of the ability of the reference polypeptide.

The term “functional variant” further includes conservatively substituted variants. The term “conservatively substituted variant” refers to a peptide having an amino acid sequence that differs from a reference peptide by one or more conservative amino acid substitutents, and maintains some or all of the activity of the reference peptide. A “conservative amino acid substitution” is a substitution of an amino acid residue with a functionally similar residue. Examples of conservative substitutions include the substitution of one non-polar (hydrophobic)residue such as isoleucine, valine, leucine or methionine for another; the substitution of one charged or polar (hydrophilic) residue for another such as between arginine and lysine, between glutamine and asparagine, between threonin and serine; the substitution of one basic residue such as lysine or arginine for another; or the substitution of one acidic residue, such as aspartic acid or glutamic acid for another; or the substitution of one aromatic residue, such as phenylalanine, tyrosine, or tryptophan for another. Such substitutions are expected to have little or no effect on the apparent molecular weight or isoelectric point of the protein or polypeptide. The phrase “conservatively substituted variant” also includes peptides wherein a residue is replaced with a chemically-derivatized residue, provided that the resulting peptide maintains some or all of the activity of the reference peptide as described herein.

The term “variant,” in connection with the polypeptides of the subject technology, further includes a functionally active polypeptide having an amino acid sequence at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 9.%, at least 94%, at least 95%, at least 96%, at least 97%, at least 98%, at least 99%, or even 100% identical to the amino acid sequence of a reference polypeptide.

The term “homologous” in all its grammatical forms and spelling variations refers to the relationship between polynucleotides or polypeptides that possess a “common evolutionary origin,” including polynucleotides or polypeptides from super families and homologous polynucleotides or proteins from different species (Recek et al., Cell 50:667, 1987). Such polynucleotides or polypeptides have sequence homology, as reflected by their sequence similarity, whether in terms of percent identity or the presence of specific amino acids or motifs at conserved positions. For example, two homologous polypeptides may have amino acid sequences that are at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90% at least 91%, at least 92%, at least 93%, at least 94%, at least 95%, at least 96%, at least 97%, at least 98%, at least 99%, or even 100% identical.

“Suitable regulatory sequences” is to be given its ordinary and customary meaning to a person of ordinary skill in the art, and is used without limitation to refer to nucleotide sequences located upstream (5′ non-coding sequences), within, or downstream (3′ non-coding sequences of a coding sequence, and which influence the transcription, RNA processing or stability, or translation of the associated coding sequence. Regulatory sequences may include promoters, translation leader sequences, introns, and polyadenylation recognition sequences.

“Promoter” is to be given its ordinary and customary meaning to a person of ordinary skill in the art, and is used without limitation to refer to a DNA sequence capable of controlling the expression of a coding sequence or functional RNA. Typically, a coding sequence is located 3′ to a promoter sequence. Promoters may be derived in their entirety from a native gene, or be composed of different elements derived from different promoters found in nature, or even comprise synthetic DNA segments. It is understood by those skilled in the art that different promoters may direct the expression of a gene in different tissues or cell types, or at different stages of development, or in response to different environmental conditions. Promoters, which cause a gene to be expressed in most cell types at most times, are commonly referred to as “constitutive promoters”. It is further recognized that since in most cases the exact boundaries of regulatory sequences have not been completely defined, DNA fragments of different lengths may have identical promoter activity.

The term “operably linked” refers to the association of nucleic acid sequences on a single nucleic acid fragment so that the function of one is affected by the other. For example, a promoter is operably linked with a coding sequence when it can affect the expression of that coding sequence (i.e., that the coding sequence is under the transcriptional control of the promoter). Coding sequences can be operably linked to regulatory sequences in sense or antisense orientation.

The term “expression” as used herein, is to be given its ordinary and customary meaning to a person of ordinary skill in the an, and is used without limitation to refer to the transcription and stable accumulation of sense (mRNA) or antisense RNA derived from the nucleic acid fragment of the subject technology.

The term “overexpress,” “overexpresses” or “overexpression” as used herein refers to higher levels of activity of a gene, e.g. transcription of the gene; higher levels of translation of mRNA into protein; and/or higher levels of production of a gene product. e.g. polypeptide, than would be in the cell in its native or control, e.g. not transformed with the particular heterologous or recombinant polypeptides being overexpressed, state. A typical example of an overexpressed gene is a gene under transcription control of another promoter as compared to the native promoter of the gene. Also, or alternatively, other changes in the control elements of a gene, such as enhancers, could be used to overexpress the particular gene. Furthermore, modifications that affect. i.e. increase, the translation of the mRNA transcribed from the gene could, alternatively or in addition, be used to achieve an overexpressed gene as used herein. These terms can also refer to an increase in the number of copies of a gene and/or an increase in the amount of mRNA and/or gene product in the cell. Overexpression can result in levels that are 25%, 50%, 100%, 200%, 500%, 1000%, 2000% or higher in the cell, as compared to control levels.

“Transformation” is to be given its ordinary and customary meaning to a person of reasonable skill in the craft, and is used without limitation to refer to the transfer of a polynucleotide into a target cell. The transferred polynucleotide can be incorporated into the genome or chromosomal DNA of a target cell, resulting in genetically stable inheritance, or it can replicate independent of the host chromosomal. Host organisms containing the transformed nucleic acid fragments are referred to as “transgenic” or “transformed”.

The terms “transformed,” “transgenic,” and “recombinant,” when used heroin in connection with host cells, are to be given their respective ordinary and customary meanings to a person of ordinary skill in the art, and are used without limitation to refer to a cell of a host organism, such as a plant or microbial cell, into which a heterologous nucleic acid molecule has been introduced. The nucleic acid molecule can be stably integrated into the genome of the host cell, or the nucleic acid molecule can be present as an extrachromosomal molecule. Such an extrachromosomal molecule car be auto-replicating. Transformed cells, tissues, or subjects are understood to encompass not only the end product of a transformation process, but also transgenic progeny thereof.

The terms “recombinant,” “heterologous,” and “exogenous,” when used herein in connection with polynucleotides, are to be given their ordinary and customary meanings to a person of ordinary skill in the art, and are used without limitation to refer to a polynucleotide (e.g., a DNA sequence or a gene) that originates from a source foreign to the particular host cell or, if from the same source, is modified from its original form. Thus, a heterologous gene in a host cell includes a gene that is endogenous to the particular host cell but has been modified through, for example, the use of site-directed mutagenesis or other recombinant techniques. The terms also includc non-naturally occurring multiple copies of a naturally occurring DNA sequence. Thus, the term refer to a DNA segment that is foreign or heterologous to the cell, or homologous to the cell but in a position or form within the host cell in which the element is not ordinarily found.

Similarly, the terms “recombinant,” “heterologous,” and “exogenous,” when used herein in connection with a)polypeptide or amino acid sequence, means a polypeptide or amino acid sequence that originates from, a source foreign to the particular host cell or, if from the same source, is modified from its original form. Thus, recombinant DNA segments can be expressed in a host cell to produce a recombinant polypeptide.

An “endogenous”, “native” or “wild type” nucleic acid, nucleotide sequence, polypeptide or amino acid sequence refers to a naturally occurring or endogenous nucleic acid, nucleotide sequence, polypeptide or amino acid sequence. Thus, for example, a “wild type mRNA” is an mRNA that is naturally occurring in our endogenous to the organism. A “homologous” nucleic acid sequence is a nucleotide sequence naturally associated with a host cell into which it is introduced.

The terms “plasmid,” “vector,” and “cassette” are to be given their respective ordinary and customary meanings to a person of ordinary skill in the art, and are used without limitation to refer to an extra chromosomal element often carrying genes which are not part of the central metabolism of the cell, and usually in the form of circular double-stranded DNA molecules. Such elements may be autonomously replicating sequences, genome integrating sequences, phage or nucleotide sequences, linear or circular, of a single- or double-stranded DNA or RNA, derived from any source, in which a number of nucleotide sequences have been joined or recombined into a unique construction which is capable of introducing a promoter fragment and DNA sequence for a selected gene product along with appropriate 3 untranslated sequence into a cell. “Transformation cassette” refers to a specific vector containing a foreign gene and having elements in addition to the foreign gene that facilitate transformation of a particular host cell. “Expression cassette” refers to a specific vector containing a foreign gene and having elements in addition to the foreign gene that allow for enhanced expression of that gene in a foreign host.

As used herein, the term “increased copy number” means at least one extra copy of at least the polypeptide coding sequence of a given gene is present in a recombinant cell as compared to the number of copies of the same gene in a parental cell, e.g., a wild-type cell, from which the recombinant cell is derived. For example, the recombinant cell may contain 1, 2, 3, 4, 5, 10, or more extra copies of at least the polypeptide codiing sequence of at given gene relative to the number of copies of that same gene in the parental cell. The extra copies of a given gene may be integrated into the genome of the recombinant cell or may be present on one or more autonomously replicating vectors or plasmids present in the recombinant cell.

As used herein, the term “sequence identity” refers to the extent to which two optimally aligned polynucleotide or peptide sequences are invariant throughout a window of alignment of components, e.g., nucleotides or amino acids. An “identity fraction” for aligned segments of a test sequence and a reference sequence is the number of identical components which are shared by the two aligned sequences divided by the total number of components in reference sequence segment, i.e., the entire reference sequence or a smaller defined part of the reference sequence.

As used herein, the term “percent sequence identity” or “percent identity” refers to the percentage of identical nucleotides in a linear polynucleotide sequence of a reference (“query”) polynucleotide molecule (or its complementary strand) as compared to a test (“subject”) polynucleotide molecule (or its complementary strand) when the two sequences are optimally aligned (with appropriate nucleotide insertions, deletions, or gaps totaling less than 20 percent of the reference sequence over the window of comparison). Optimal alignment of sequences for aligning a comparison window are well known to those skilled in the art and may be conducted by tools such as the local homology algorithm of Smith and Waterman, the homology alignment algorithm of Needleman and Wunsch, the search for similarity method of Pearson and Lipman, and preferably by computerized implementations of those algorithms such as GAP, BESTFIT, FASTA, and TFASTA available as part of the GCG(registered trademark) Wisconsin Package(registered trademark) (Accelrys Inc., Burlington, MA). An “identity fraction” for aligned segments of a test sequence and a reference sequence is the number of identical components which are shared by the two aligned sequences divided by the total number of components in the reference sequence segment, i.e., the entire reference sequence or a smaller defined part of the reference sequence. Percent sequence identity is represented as the identity fraction multiplied by 100. The comparison of one or more polynucleotide sequences may be to a full-length polynucleotide sequence or a portion thereof, or to a longer polynucleotide sequence. For purposes of this disclosure “percent identity” may also be determined using BLASTX version 2.0 for translated nucleotide sequences and BLASTN version 2.0 for polynucleotide sequences.

The percent of sequence identity is preferably determined using the “Best Fit” or “Gap” program of the Sequence Analysis Software Package (trademark) (Version 10; Genetics Computer Group, Inc., Madison, WI). “Gap” utilizes the algorithm of Needleman and Wunsch (Needleman and Wunsch. Journal of Molecular Biology 48:443-453, 1970) to find the alignment of two sequences that maximizes the number of matches and minimizes the number of gaps. “BestFit” performs an optimal alignment of the best segment of similarity between two sequences and inserts gaps to maximize the number of matches using the local homology algorithm of Smith and Waterman (Smith and Waterman, Advances in Applied Mathematics, 2:482-489, 1981, Smith et al., Nucleic Acids Research 11:2205-2220, 1983). The percent identity is most preferably determined using the “Best Fit” program.

Useful methods for determining sequence identity are also disclosed in the Basic Local Alignment Search Tool (BLAST) programs which are publicly available from National Center Biotechnology Information (NCBI) at the National Library of Medicine, National Institute of Health, Bethesda, Md. 20894; see, BLAST Manual, Altschul et al., NCBI, NLM, NIH; Altschul et al., J. Mol. Biol. 215:403-410(1990); version 2.0 or higher of BLAST programs allows the introduction of gaps (deletions and insertions) into alignments; for peptide sequence BLASTX can be used to determine sequence identity; and, for polynucleotide sequence BLASTN can be used to determine sequence identity.

As used herein, the term “substantial percent sequence identity” refers to a percent sequence identity of at least about 70% sequence identity, at least about 80% sequence identity, at least about 85% identity, at least about 90% sequence identity, or even greater sequence identity, such as about 98% or about 99% sequence identity. Thus, one embodiment of the invention is a polynucleotide molecule that has at least about 70% sequence identity, at least about 80% sequence identity, at least about 85% identity, at least about 90% sequence identity, or even greater sequence identity, such as about 98% or about 99% sequence identity with a polynucleotide sequence described herein. Polynucleotide molecules encoding proteins that have the activity of the gs5119 sequence of the current disclosure are capable of directing the production of a variety of jasmonic compounds and have a substantial percent sequence identity to the polynucleotide sequences provided herein and are encompassed within the scope of this disclosure.

As used herein, the term “identity” is the fraction of amino acids that are the same between a pair of sequences after an alignment of the sequences (which can be done using only sequence information or structural information or some other information, but usually it is based on sequence information alone), and the term “similarity” refers to the score assigned based on an alignment using some similarity matrix. The similarity index can be any one of the following BLOSUM62, PAM250, or GONNET, or any matrix used by one skilled in the art for the sequence alignment of proteins.

Identity is the degree of correspondence between two sub-sequences (no gaps between the sequences). An identity of 25% or higher implies similarity of function, while 0.18-25% implies similarity of structure or function. Keep in mind that two completely unrelated or random sequences (that are greater than 100 residues) can have higher than 20% identity. Similarity is the degree of resemblance between two sequences when they are compared. This is dependent on their identity.

As is evident from the present description, certain aspects of the present disclosure are not limited by the particular details of the examples illustrated herein, and it is therefore contemplated that other modifications and applications, or equivalents thereof, will occur to those skilled in the art. It is accordingly intended that the claims shall cover all such modifications and applications that do not depart from the spirit and scope of the present disclosure.

Moreover, unless defined otherwise, all technical and scientific terms used herein have the same meaning as commonly understood by one of ordinary skill in the art to which the disclosure belongs. Although any methods and materials similar to or equivalent to or those described herein can be used in the practice or testing of the present disclosure, the preferred methods and materials are described above.

### Metabolically Engineered Cell for Jasmonic Acid Biosynthesis:

In one aspect, the present disclosure provides a host cell having an operative metabolic pathway that includes at least one enzymatic activity. The pathway produces 12-oxophytodienoic acid (12-OPDA) which is then reduced into 3-oxo-2-(cis-2′-pentenyl)-cyclopentane-1-octanoic acid (OPC8). Such a cell may be naturally occurring but in certain embodiments is a recombinant cell produced by genetic engineering.

In representative embodiments, the OPC8 is produced in a reaction where endogenous 12-OPDA is a substrate and the react ion is catalyzed by a polypeptide having OPR activity. The polypeptide may be a “gs5119-like protein” (or “gs5119 protein” or “gs5119”) comprising the amino acid sequence a; set forth in SEQ ID NO:1 or a variant thereof having OPR activity and comprising an amino acid sequence having at least 90% (e.g., at least 90%, at least 91%, at least 92%, at least 93%, at least 94%, at least 95%, at least 96%, at least 97%, at least 98%, at least 99%, or 100%) identity to SEQ ID NO:1. In aspects of these embodiments, the variant polypeptide has the same or substantially the same OPR activity as the gs5119 described herein, e.g., an enzyme comprising the amino acid sequence of SEQ ID NO:1.

The polypeptide having CPR activity is overexpressed relative to a corresponding parental host cell from which the recombinant host cell is derived. In some embodiments, the overexpression may be achieved by genetically engineering the host cell to include one or more ectopic copies of a native gene coding, for the polypeptide having OPR activity. In further, non-exclusive embodiments, the overexpressing the polypeptide having OPR activity is effected by replacing a native promoter of a gene expressing the polypeptide having OPR activity with U promoter having a higher level of expression than the promoter native to the host cell. Exemplary higher-level promoters include ToxA and ToxB from Pyrenophora tritici-repentis, although any suitable promoters for high level recombinant expression in fungal host cells may serve this role. Alternatively, or in conjunction with the foregoing approaches, overexpressing of the polypeptide having OPR activity may be achieved by recombinantly introducing into the host ccli at least one copy of an exogenous polynucleotide sequence that is not native to the host cells and encodes the polypeptide having OPR activity.

The nucleic acid coding for the polypeptide having OPR activity may include a polynucleotide sequence coding for a gs5119-like polypeptide. The polynucleotide sequence, also known as “gs5119-encoding poly nucleotide” or “gs5.119 polynucleotide” may be, for example, the sequence as forth in SEQ ID NO: 2 or a variant thereof, such as the codon-optimized sequence as set forth in SEQ ID NO: 3. Therefore, in a number of embodiments, the disclosure provides a nucleic acid comprising a nucleotide sequence having at least 90% (e.g., at least 90%, at least 91%, at least 92%, at least 93%, at least 94%, at least 95%, at least 96%, at least 97%, at least 98%, at least 99%, or 100%) identity to SEQ ID NO: 2. Also provided is a nucleic acid comprising a nucleotide sequence having at least 90% (e.g., at least 90%, it least 91%, at least 92%, at least 93%, at least 94%, at least 95%, at least 96%, at least 97%, at least 98%, at least 99%, or 100%) identity to SEQ ID NO: 3. In some embodiments, the nucleotide sequence encodes a polypeptide including enzyme gs5119, i.e., an enzyme comprising the amino acid sequence of SEQ ID NO: 1.

In certain embodiments of this aspect, the host cell may be a bacterium. Typical bacterial genera include: Escherichia; Salmonella; Bacillus; Acinetobacter; Corynebacterium; Methylosinus; Methylomonas; Rhodococcus; Pseudomonas; Rhodobacter; Synechocystis; Aspergillus; Arthrobotrys; Brevibacteria; Microbacterium; Arthrobacter; Citrobacter; Klebsiella; Pantoea; Salmonella; Corynebacterium, and Clostridium.

In further embodiments, the host cell is a eukaryotic cell selected from the group consisting of a yeast cell, a filamentous fungus cell, a plant cell, and an animal cell.

An aspect of the embodiments relates to genetically modified filamentous fungal cells capable of producing jasmonates. Typical fungal cells include species of the genera Lasiodiplodia, Rhizopus, Fusidium, Gibberella, Trichoderma, Hypocrea, Aspergillus, Fusarium, Penicillium, Neurospora, Chaetomium, Acremonium, Glomerella, Myceliophthora, Sporotrichum, Thielavia, Chrysosporium, Corynascus, Ctenomyces, Verticillium, Cordyceps, Nectria, and Magnaporthe including anamorphs and teleomorphs thereof, as well as recognized synonymous genera.

The phrase “a fungal species belonging to the genus Lasiodiplodia” can mean that the fungus is classified as the genus Lasiodiplodia according to the classification known to a person skilled in the art of mycology. Specifically, those classified into the group Lasiodiplodia according to the taxonomy used by the NCBI (National Center for Biotechnology Information) database (www.ncbi.nlm.nih.gov/Taxonomy/Browser/wwwtax.cgi) can be used. Example of the fungus belonging to the genus Lasiodiplodia include, but are not limited to, Lasiodiplodia abnormis, Lasiodiplodia citri, Lasiodiplodia citricola, Lasiodiplodia erassispora, Lasiodiplodia fiorii, Lasiodiplodia frezaliana, Lasiodiplodia gilanensis, Lasiodiplodia gonubiensis, Lasiodiplodia hormozganensis, Lasiodiplodia iraniensis, Lasiodiplodia margaritacea, Lasiodiplodia missouriana, Lasiodiplodia paraphysaria, Lasiodiplodia parva, Lasiodiplodia plurivora, Lasiodiplodia pseudotheobromae, Lasiodiplodia ricini, Lasiodiplodia rubropurpurea, Lasiodiplodia theobromae, Lasiodiplodia thomasiana, Lasiodiplodia undulata, Lasiodiplodia venezuelensis, Lasiodiplodia viticola. Fungal species belonging to other genera include, but are not limited to, T. reesei, H. jecorina, A. niger, A. fumigatus, A, oryzae, A. nidulans, F. oxysporum, N. crassa, C, thermophilum, A, thermophilum, O. graminicola, M. thermophila, S, thermophile, T. terestris, T. heterothallica, C, thermophile, V. dahlia, C. militaris, N. heamatococca, or M. oryzae.

In another aspect of the embodiments, the host cell is a cell isolated from plants selected from the group consisting of soybean; rapeseed; sunflower; cotton; con; tobacco; alfalfa; wheat; barley; oats; sorghum; rice; broccoli; cauliflower; cabbage; parsnips; melons; carrots; celery; parsley; tomatoes; potatoes; strawberries; peanuts; grapes; grass seed crops; sugar beets; sugar cane; beans; peas; rye; flax; hardwood trees; softwood trees; forage grasses; Arabidopsis thaliana; rice (Oryza sativa); Hordeum yulgare; switchgrass (Panicum vigratum); Brachypodium spp.; Brassica spp.; and Crambe abyssinica.

In some embodiments, the host cell is genetically modified to overexpress a polypeptide having OPR activity relative to the parent, unmodified cell, e.g., a wild-type host cell, wherein the polypeptide comprises an amino acid sequence having at least 90%, at least 91%, at least 92%, at least 93%, at least 94%, at least 95%, at least 96%, at least 97%, at least 98%, at least 99%, or even 100% identity to the amino acid sequence as set forth in SEQ ID NO:1. The protein having OPR like protein may be homologous or heterologous with respect to the host cell. For the purposes herein, in certain embodiments the homologous polypeptide having OPR activity is encoded by a polynucleotide sequence that naturally occurs in, or is isolated or derived from, the same or taxonomically equivalent taxonomic species as the host cell. Furthermore, as is recognizes by one of skill in the art, a homologous protein may contain one or more insertions, deletions and substitutions and still be considered to be “derived from” the same species as the wild-type host coll. Such one or more insertions, deletions and substitutions may result in increased or decreased expression or activity of the homologous polypeptide. Similarly, a polynucleotide encoding a homologous polypeptide having OPR activity may contain one or more insertions, deletions and substitutions (including substitutions that optimize codon usage without altering the sequence of the encoded protein).

A heterologous polypeptide having OPR activity is encoded by a polynucleotide sequence that naturally occurs in, or is isolated or derived from, a different taxonomic species from the host cell. Furthermore, as is recognized by one of skill in the art, a heterologous polypeptide having OPR activity may contain one or more insertions, deletions and substitutions and still be considered to be “derived from” a different taxonomic species from the host cell. Such one or more insertions, deletions and substitutions may result in increased or decreased expression or activity of the heterologous polypeptide having OPR activity. Similarly, a polynucleotide encoding a heterologous polypeptide having OPR activity may contain one or more insertions, deletions and substitutions including substitutions that optimize codon usage without altering the sequence of the encoded protein).

As used herein, in respect of polynucleotide sequences, “derived from” refers to the isolation of a target polynucleotide sequence using one or more molecular biology techniques known to those of skill in the art including, but not limited to, reverse translation of a polypeptide or amino acid sequence, cloning, sub-cloning, amplification by PCR, in vitro synthesis, and the like. Furthermore, as is recognized by one of skill in the art, a polynucleotide sequence that is derived from a target polynucleotide sequence may be modified by one or more insertions, deletions and substitutions and still be considered to be “derived from” that target nucleotide sequence. Such one or more insertions, deletions and substitutions may result in increased or decreased expression or activity of the protein of interest encoded by the polynucleotide sequence and may be located within a promoter sequence, the 5′ or 3′ untranslated regions, or within the coding region for the protein of interest.

As used herein with respect to polynucleotide sequences, “isolated” or “isolation” means altered from its natural state by virtue of separating the nucleic acid sequence from some or all of the naturally-occurring nucleic acid sequences with which it is associated in nature.

In some embodiments, the host cell may be genetically modified by transformation of the host cell with a gs5119 genetic construct. As used herein. “gs5119 genetic construct” refers to an isolated polynucleotide comprising elements necessary for increasing the expression of a gs5119-like protein. These elements may include, but are not limited to, a polynucleotide sequence encoding a gs5119-like protein (coding sequence), a promoter operably linked to the coding sequence and comprising polynucleotide sequences that direct the transcription and translation of the coding sequence. The recombinant host cell of the present invention may further comprise one or more genetic constructs that direct the production and secretion of one or more homologous or heterologous polypeptides having OPR activity, for example a gs5119 or gs5119-like protein. Such constructs comprise polynucleotide elements including, but not limited to, a coding sequence for the polypeptide, a promoter operably linked to the coding sequence and comprising a polynucleotide sequence that directs the transcription of the coding region, and a sequence encoding a secretion signal peptide operably linked to the coding sequence, as well as targeting polynucleotide sequences that direct homologous recombination of the construct into the genome of the host cell. The terms “secretion signal peptide”, “secretion signal” and “signal peptide” refer to any sequence of nucleotides and/or amino acids which may participate in the secretion of the mature or precursor forms of a secreted protein. The signal sequence may be endogenous or exogenous with respect to the host cell. The signal sequence may be that normally associated with the protein of interest or a gene encoding another secreted protein. The signal sequence may also be a “hybrid signal sequence” containing partial sequences from two or more genes encoding secreted proteins.

As understood by one of ordinary skill in the art, the coding sequence, promoter, and/or secretion signal may be derived from the parental host cell, from a different organism, and/or be synthesized in vitro. For example, the promoter and secretion signal may be derived from one or more genes encoding proteins that are highly expressed and secreted when a parental host cell is grown in a fermentation process such as that defined below, for example, gene(s) typically highly expressed in filamentous fungi. These polynucleotide elements may also be altered or engineered by replacement, substitution, addition, or elimination of one or more nucleic acids relative to a naturally-occurring polynucleotide. However, it should be understood that the practice of the present invention is not limited by the choice of promoter in the gs5119 genetic construct or by the choice of promoter and secretion signal in genetic constructs expressing gs5119-like enzymes.

The genetic constructs described above may contain a selectable marker for identification of transformed host cells. The selectable marker may be present on the genetic construct or the selectable marker may be a separate isolated polynucleotide that is cotransformed with the genetic construct. Choices of selectable markers are well known to those skilled in the art and include genes (synthetic or natural) that confer to the transformed cells the ability to utilize a metabolite that is not normally metabolized by the microbe (e.g., the A. nidulans amdS gene encoding acetamidase and conferring the ability to grow on acetamide as the sole nitrogen source) or antibiotic resistance (e.g., the Escherichia coli hph gene encoding hygr mycin-beta-phosphotransferase and conferring resistance to hygromycin). Alternatively, if the host cell expresses little or none of a chosen marker activity, then the corresponding gene may be used as a marker. Examples of such markers include trp, pyr4, pyrG, argB, leu, and the like. The corresponding host strain would therefore have to be lacking a functional gene corresponding to the marker chosen. i.e., lacking in the expression of trp, pyr, arg, leu and the like.

A genetic construct may contain a transcriptional terminator that is functional in the host cell, as would be known to one of skill in the art. The transcriptional terminator may be positioned immediately downstream of a coding sequence. The practice of the invention is not constrained by the choice of transcriptional terminator that iv sufficient to direct the termination of transcription in the host cell.

A genetic construct may contain additional polynucleotide sequences between the various sequence elements as described herein. These sequences, which may be natural or synthetic, may result in the addition of one or more of the amino acids to the protein encoded by the construct. The practice of the invention is not constrained by the presence of additional polynucleotide sequences between the various sequence elements of the genetic constructs present in the host cell.

As disclosed above, some embodiments relate to recombinant filamentous fungal host cells. Methods of introducing a genetic construct into a fungal cell are familiar to those skilled in the art and include, but are not limited to, Agrobacterium tumefaciens-mediated transformation, calcium chloride treatment of fungal protoplasts to weaken the cell membranes, addition of polyethylene glycol to allow for fusion of cell membranes, depolarization of cell membranes by electroporation, or shooting the construct through the cell wall and membranes via microprojectile bombardment with a particle gun. The practice of the present invention is not constrained by the method of introducing the genetic constructs into the fungal cell.

It will be understood that a recombinant fungal cell a, presented herein encompasses both the perfect and imperfect states, and other taxonomic equivalents, e.g., anamorphs and teleomorphs, regardless of the species name by which they are known. Further examples of taxonomic equivalents can be found, for example, in Cannon, Mycopathologica 111:75-83, 1990; Moustafa et al., Persoonia 14:173-175, 1990; Stalpers, Stud. Mycol. 24, 1984; Upadhyay et al., Mycopathologia 87:71-80, 1984; Guarro et al., Mycotaxon 23: 419-421, 1985; Awao et al., Mycotaxon 16:436-440, 1983; von Klopotek, Arch. Microbiol. 98:365-369, 1974; and Long et al., 1994, ATCC Names of Industrial Fungi, ATCC, Rockville Md. Those skilled in the art will readily recognize the identity of appropriate equivalents. Accordingly, it will be understood that, unless otherwise stated, the use of a particular genus and/or species designation in the present disclosure also refers to genera and species that are related by anamorphic or teleomorphic relationship, genera and species that are recognized as synonymous, as well as those that have been or may be reclassified into one of the claimed genera or species in the future.

In plant cells, the expression vectors of the subject technology can include a coding region operably linked to promoters capable of directing expression of the recombinant polypeptide of the subject technology in the desired tissues at the desired stage of development. For reasons of convenience, the polynucleotides to be expressed may comprise promoter sequences and translation leader sequences derived from the same polynucleotide. 3′ non-coding sequences encoding transcription termination signals should also be present. The expression vectors may also comprise one or more introns to facilitate polynucleotide expression.

For plant host cells, any combination of any promoter and any terminator capable of inducing expression of a coding region may be used in the vector sequences of the subject technology. Some suitable examples of promoters and terminators include those from nopaline synthase (nos), octopine synthase (ocs) and cauliflower mosaic virus CaMV) genes. One type of efficient plant promoter that may be used is a high-level plant promoter. Such promoters, in operable linkage with an expression vector of the subject technology should be capable of promoting the expression of the vector. High level plant promoters that may be used in the subject technology include the promoter of the small subunit (ss) of the ribulose-1, 5-bisphosphate carboxylase for example from soybean (Berry-Lowe et al., J. Molecular and App. Gen., 1:483 498 (1982), the entirety of which is hereby incorporated herein to the extent it is consistent herewith), and the promoter of the chlorophyll alb binding protein. These two promoters are known to be light-induced in plant cells (see, for example, Genetic Engineering of Plants, an Agricultural Perspective, A. Cashmore, Plenum, N.Y. (1983), pages 29 3N. Coruzzi, G. et al., The Journal of Biological Chemistry, 258: 1399 (1983), and Dunsmuir, P. et al., Journal of Molecular and Applied Genetics, 2:285 (1983), each of which is hereby incorporated herein by reference to the extent they are consistent herewith).

### Synthetic Biology

Standard recombinant DNA and molecular cloning techniques used here are well known in the art and are described, for example, by Sambrook, J., Fritsch, E. F. and Maniatis, T. Molecular Cloning: A Laboratory Manual, 2nd ed.; Cold Spring Harbor Laboratory: Cold Spring Harbor, N.Y., 1989 (hereinafter “Maniatis”); and by Silhavy, T. J., Bennan, M. L. and Enquist, L. W. Experiments with Gene Fusions; Cold Spring Harbor Laboratory: Cold Spring Harbor, N.Y., 1984; and by Ausubel, F. M. et al., In Current Protocols in Molecular Biology, published by Greene Publishing and Wiley-Interscience, 1987; (the entirety of each of which is hereby incorporated herein by reference).

### Production Systems

Expression of proteins in recombinant host cells is often carried out with vectors containing constitutive or inducible promoters directing the expression of one or more recombinant proteins.

Fusion vectors add a number or amino acids to a protein encoded therein, usually to the amino terminus of the recombinant protein. Such fusion vectors typically serve three purposes: (1) to increase expression of recombinant protein; 42) to increase the solubility of the recombinant protein; and (3) to aid in the purification of the recombinant protein by acting as a ligand in affinity purification. Often, a proteolytic cleavage site is introduced at the junction of the fusion moiety and the recombinant protein to enable separation of the recombinant protein from the fusion moiety subsequent to purification of the fusion protein. Such vectors are within the scope of the present disclosure.

In an embodiment, the expression vector includes those genetic elements for expression of the recombinant polypeptide in bacterial cells or other microbes. The elements for transcription and translation in the microbe cell may include a promoter, a coding region for the protein complex, and a transcriptional terminator.

A person of ordinary skill in the art will be aware of the molecular biology techniques available for the preparation of expression vectors. The polynucleotide used for incorporation into the expression vector of the subject technology, as described above, can be prepared by routine techniques such as polymerase chain reaction (PCR).

Several molecular biology techniques have been developed to operably link DNA to vectors via complementary cohesive termini. In one embodiment, complementary homopolymer tracts can be added to the nucleic acid molecule to be inserted into the vector DNA. The vector and nucleic acid molecule are then joined by hydrogen bonding between the complementary homopolymeric tails to form recombinant DNA molecules.

In alternative embodiments, synthetic linkers containing one or more restriction sites provide are used to operably link the polynucleotide of the subject technology to the expression vector. In an embodiment, the polynucleotide is generated by restriction endonuclease digestion. In one instance, the nucleic acid molecule is treated with bacteriophage T4 DNA polymerase or E. coli DNA polymerase I, enzymes that remove protruding. 3′-single-stranded tenmini with their 3-5′-exonucleolytic activities, and fill in recessed 3-ends with their polymerizing activities, thereby generating blunt ended DNA segments. The blunt-ended segments are then incubated with a large molar excess of linker molecules in the presence of an enzyme that can catalyze the ligation of blunt-ended DNA molecules, such as bacteriophage T4 DNA ligase. Thus, the product of the react ion is a polynucleotide carrying polymeric linker sequences at its ends. These polynucleotides are when cleaved with the appropriate restriction enzyme and ligated to an expression vector that has been cleaved with an enzyme that produces termini compatible with these of the polynucleotide.

Alternatively, a vector having ligation-independent cloning (LIC) sites can be employed. The required PCR amplified polynucleotide can then be cloned into the LIC vector without restriction digest or ligation (Aslanidis and de Jong, Nucl. Acid. Res. 18 6069-74, (1990), Haun, et al, Biotechniques 13, 515-18 (1992), which is incorporated herein by reference to the extent it is consistent herewith).

In an embodiment, to isolate and/or modify the polynucleotide of interest for insertion into the chosen plasmid, ii is suitably to use PCR. Appropriate primers for use in PCR preparation of the sequence can be designed to isolate the required coding region of the nucleic acid molecule, add restriction endonuclease or LIC sites, place the coding region in the desired reading frame.

In an embodiment, a polynucleotide for incorporation into an expression vector of the subject technology is prepared using PCR using appropriate oligonucleotide primers. The coding region is amplified, while the primers themselves become incorporated into the amplified sequence product. In an embodiment, the amplification primers contain restriction endonuclease recognition sites, which allow the amplified sequence product to be cloned into an appropriate vector.

The expression vectors can be introduced into host cells, for example bacteria or other microbes such as yeasts or fungi, by conventional transformation or transfection techniques. Transformation of appropriate cells with an expression vector of the subject technology is accomplished by methods known in the art and typically depends on both the type of vector and cell. Suitable techniques include calcium phosphate or calcium chloride co-precipitation, DEAE-dextran mediated transfection, lipofection, chemoporation or electroporation.

Microbial host ccli expression systems and expression vectors containing regulatory sequences that direct high-level expression of foreign proteins are well known to those skilled in the art. Any of these may be used to construct vectors for expression of the recombinant polypeptide of the subjection technology in a microbial host cell. These vectors may then be introduced into appropriate microorganisms via transformation to allow for high level expression of the recombinant polypeptide of the subject technology.

Vectors or cassettes useful for the transformation of suitable microbial host cells are well known in the art. Typically, the vector or cassette contains sequences directing transcription and translational of the relevant polyglucoside, a selectable marker, and sequences allowing autonomous replication or chromosomal integration. Suitable vectors comprise a region 5′ of the polynucleotide which harbors transcriptional initiation controls and a region 3 of the DNA fragment which controls transcriptional termination. It is preferred for both control regions to be derived from genes homologous to the transformed host cell, although it is to be understood that such control regions need not be derived from the genes native to the specific species chosen as a host.

Initiation control regions or promoters, which are useful to drive expression of the recombinant polypeptide in the desired microbial host cell are numerous and familiar to those skilled in the art. Virtually any promoter capable of driving these genes is suitable for the subject technology including but not limited to CYC1, HIS3, GALI, GALIO, ADHI, PGK, PH05, GAPDH, ADCI, TRPI, URA3, LEU2, ENO, TPI (useful for expression in Saccharomyces); AOXI (useful for expression in Pichia); lac, trp, JPL, IPR, T7, tac, and trc (useful for expression in Escherichia coli); and ToxA and ToxB (useful for expression in filamentous fungi).

Successfully transformed cells, that is, those cells containing the expression vector, can be identified by techniques well known in the art. For example, cells transfected with an expression vector of the subject technology can be cultured to produce polypeptides described herein. Cells can be examined for the presence of the expression vector DNA by techniques well known in the art. The transformed host cells may contain a single copy of the expression vector described previously, or alternatively, multiple copies of the expression vector.

### Biosynthesis of Jasmonates

In a further aspect, the present invention comprises cultivating a recombinant host cell comprising a metabolic pathway producing jasmonic acid, wherein the hot cell overexpresses a polypeptide having 12-oxophytodienoate reductase (OPR) activity relative to a corresponding parental host cell. The recombinant host cell may be cultivated in a submerged liquid fed-batch or continuous culture.

As used herein, the terms “cultivating” and “culturing” refer to growing a cell culture, for example a population of microbial cells, under suitable conditions in a liquid or solid medium. The culturing may be carried out using conventional fermentation equipment suitable for such purpose (e.g., shake flasks, fermentation tanks, and bioreactors).

A “submerged liquid culture”, as defined herein, is a cell culture in which the cells are suspended, or significantly suspended, in a liquid medium containing nutrients required for maintaining the viability of the cells. The culture is generally agitated at a sufficient rate to ensure distribution of the cells throughout the medium. The agitation rate is typically also selected to prevent formation of concentration gradients of nutrients.

In a “batch process” or “batch fermentation”, all the necessary culture and media components, with the exception of oxygen for aerobic processes, are placed in a reactor at the smart of the operation and the fermentation is allowed to proceed until completion, at which point the product is withdrawn from the reactor.

In a “red batch process” or “fed-batch fermentation”, the culture is ted continuously or sequentially with one or more media components without the removal of the culture fluid.

In a “continuous process” or “continuous fermentation”, fresh medium is supplied and culture fluid is removed continuously at volumetrically equal, or substantially equal, rates to maintain the culture at a steady growth rate, in reference to continuous processes, “steady state” refers to a state in which the concentration of reactants does not vary appreciably, and “quasi-steady state” refers to a state in which, subsequent to the initiation of the reaction, the concentration of reactants fluctuates within a range consistent with normal operation of the continuous hydrolysis process. Continuous fermentation process may also be referred to as CSTR (continuous stirred-tank reactor) fermentations. One example of a continuous fermentation process is a chemostat, in which the growth rate of the microorganism is controlled by the supply of one limiting nutrient in the medium.

In the fermentation processes of the present invention, the recombinant host cell may be first cultured in a batch fermentation typically containing a non-inducing carbon source. Upon completion of the batch fermentation, which is typically identified by the depletion of essentially all of the available carbon source. For example, when the concentration of the carbon source in the culture filtrate is no more than 1 g/L, the recombinant host cell is cultured in a fed-batch, continuous or combined fed-hatch and continuous submerged liquid culture.

Fed-batch and continuous processes are typically carried out in one or more bioreactors. Typical bioreactors used for cell culture fermentation processes include, but are not limited to, mechanically agitated vessels or those with other means of agitation (such as air injection). Bioreactors may be temperature and pH-controlled. Typically, there are means provided to clean the reactor in place. Means may also be provided to sanitize or sterilize the bioreactor prior to introduction of the target organism so as to minimize or prevent competition for carbon sources from other organisms. Bioreactors may be constructed from many materials, but most often are of glass or stainless steel. Provisions are generally made for sampling (in a manner that prevents or minimizes the introduction of undesirable competing organisms). Means to obtain other measurements are often provided (e.g., ports and probes to measure dissolved oxygen concentration or concentration of other solutes such us ammonium ions). The practice of the invention is not limited by the choice of bioreactor(s).

In the fermentation processes of the present invention, the fed-batch, continuous or combined fed-batch and continuous submerged liquid culture is provided with a feed solution containing a carbon source. In some embodiments, the carbon source consists of one or more carbohydrate. As used herein, the term “carbon source” refers to a carbon containing substance that provides the major part of the carbon required for growth of, and production of jasmonates by a parental or recombinant cell culture. For the purposes herein, a carbon source may be one or more carbohydrate, a non-carbohydrate substance such as a sugar alcohol, organic acid, or alcohol, or combinations thereof. However, for the purposes herein, organic nitrogen sources that may be provided to the cell culture may or may not be considered carbon sources.

In the fermentation process of the present invention, the feed solution may contain one or more additional components, such as nitrogen sources, vitamins. minerals and salts required for growth of the fungal cell as in known to one of skill in the are. Nitrogen sources may be inorganic and/or organic in nature and include, but are not limited to, one or more amino acids, peptides and proteins, in pure or raw form (e.g., corn steep liquor), any number of protein hydrolysates (peptone, tryptone, casamino acids), yeast extract, ammonia, ammonium hydroxide, ammonium salts, urea, nitrate and combinations thereof. The practice of the fermentation process of the present invention is not limited by the additional components of the feed solution.

The feed solution is provided to the fermentation process at a rate, the feed rate or “carbon addition rate” or “CAR” (measured as g carbon per liter per hour). In an exemplary fermentation process according to the present invention, the feed solution may be provided to a fed-batch culture at a carbon addition rate of from about 0.2 to about 4 g carbon/L culture/h or any rate therebetween, for example 0.2, 0.3, 0.4, 0.6, 0.8, 1.0, 1.2, 1.4, 1.6, 1.8, 2.0, 2.2, 2.5, 3.0, 3.5, and 4.0 g carbon/L culture/h or any rate therebetween. Alternatively, the feed solution may be provided to a continuous culture at a dilution rate of from about 0.001 to 0.1 h, or any dilution rate therebetween, for example at about 0.001, 0.005, 0.01, 0.02, 0.03, 0.04, 0.05, 0.06, 0.07, 0.08, 0.09, 0.1 h⁻¹, or any dilution rate therebetween.

The fermentation processes of the present invention may be carried at a temperature from about 20° C. to about 55° C., or any temperature therebetween, for example from about 30° C. to about 45° C. or any temperature therebetween, or from 20, 22, 25, 28, 30, 32, 35, 38, 40, 42, 45, 48, 50° C., 55° C. or any temperature therebetween.

The fermentation processes of the present invention may be carried out at a pH1 from about 2.5 to 8.5, or any pH therebetween, for example from about pH 3.5 to pH 7.0, or any pH therebetween, for example from about pH 2.5, 3.0, 3.2, 3.5, 3.8, 4.0, 4.2, 4.5, 4.8, 5.0, 5.2, 5.4, 5.5, 5.7, 5.8, 6.0, 6.2, 6.5, 6.8, 7.0, 7.2, 7.5, 7.8, 8.0, 8.5 or any pH therebetween. The pH may be controlled by the addition of a bae, such as ammonium or sodium hydroxide, or by the addition of an acid, such as phosphoric acid.

The fermentation processes of the present invention may be carried out over a period of about 1 to 90 days, or any period therebetween, for example between 3 and 30 days, or any amount therebetween, between 3 and 8 days, or any amount therebetween, or from 1, 2, 3, 4, 5, 6, 7, 8, 9, 10, 12, 14, 16, 18, 20, 22, 24, 26, 28, 30, 40, 50, 60, 70, 80, or 90 days, or any amount therebetween.

The fermentation processes of the present invention may be performed in cultures having a volume of at least 0.5 liter, for example from about 0.5 to about 1,000,000 liters, or any amount therebetween, for example, 5 to about 400,400 liters, or any amount therebetween, 20 to about 200,000 liters, or any amount therebetween, or 2.000 to about 200.000 liter, or any amount therebetween, or from about 0.5, 1, 10, 50, 100, 200, 400, 600, 800, 1000, 2000, 4000, 6000, 8000, 10,000, 15,000, 20,000, 25,000, 30,000, 35,000, 40,000, 45,000, 50,000, 55,000, 60,000, 65,000, 70,000, 75,000, 80,000, 85,000, 90,000, 95,000, 100,000, 150,000, 200,000, 300,000, 400,000, 500,000, 750,000 or 1,000,000 liters in volume, or any amount therebetween.

The fermentation processes of the present invention may be performed aerobically, in the presence of oxygen, or anaerobically, in the absence of oxygen. For example, the process may be performed aerobically such that air or oxygen gas is provided to the submerged liquid culture at a superficial gas velocity of from about 0.001 to about 100 cm/s, or any rate therebetween, for example any rate from about 0.01 to about 20 cm/s, or any rate therebetween. An alternative parameter to measure aeration rate that is known to one of skill in the art is vessel volume per minute (vvm), in the fermentation process of the present invention, air or oxygen gas is provided to the submerged liquid culture at a rate of from about 0.5 to about 5 vvm, or any rate therebetween. Antifoaming agents (either silicone, or non-silicone based) may be added to control excessive foaming during the process as required and as is known to one of skill in the art.

As used herein, the term “specific productivity”, alternatively expressed as “qp”, refers to the rate at which one or more jasmonates are produced from a given mass of recombinant host cells. Typically, the specific productivity of a fermentation process is expressed as mg protein pet g of host cells per hour (mg protein/g cells/h) and is calculated by measuring the concentration, in mg/L, of jasmonates in culture filtrates (culture media from which the recombinant cells have been removed) and dividing by the concentration of cultured cells (in g dry weight per L) in the culture medium and dividing by the total time, in h, since the feed solution was initially provided to the culture. The fermentation processes of the present invention may also be characterized by “maximum productivity” (or “maximum qp”), which is the highest value qp calculated during the course of the fermentation process, or by “average productivity” (or “avenge qp”), which is the average of all of the values of qp calculated during the course of the fermentation process.

In some embodiments, a fermentation process according to the present invention in which the one or more jasmonates is produced from a culture of cells overexpressing a polypeptide having 12-oxophytodienoate reductase (OPR) activity relative to a corresponding parental host cell, exhibits at least a 50% increase in maximum specific productivity (qp) relative to that exhibited by an equivalent process utilizing a parental cell from which the recombinant host cell is derived. For example, such fermentation process may exhibit at least a 50%, 60%, 70%, 80%, 90%, 100%, 150%, 200%, 300%, 400%, 500% or higher increase in maximum specific productivity (qp) relative to that exhibited by an equivalent process utilizing a parental host cell from which the recombinant host cell is derived.

As used herein, the terms “equivalent fermentation process” or “equivalent process”, refer to a fermentation process in which a parental fungal cell is cultural under identical or nearly identical conditions of medium composition, time, cell density, temperature, and pH, as those used to culture an isolated fungal cell derived from that parental fungal cell.

After cultivation, products such as jasmonic acid may be extracted directly from the liquid medium. In addition, solids such as cells may be removed from the medium by centrifugation or membrane filtration, and the products may be collected and purified by ion-exchange, concentration, distillation, and crystallization methods.

Although the foregoing invention has been described in some detail by way of illustration and example for purposes of understanding, it will be apparent to these skilled in the art that certain changes and modifications may be practiced. Therefore, the description and examples should not be construed as limiting the scope of the invention, which is delineated by the appended claims.

Unless defined otherwise, all technical and scientific terms used herein have the same meaning as commonly understood by one of ordinary skill in the art to which the disclosure belongs. Although any methods and materials similar to or equivalent to those described herein can be used in the practice or testing of the present disclosure, the preferred materials and methods are described below.

The disclosure will be more fully understood upon consideration of the following non-limiting Examples. It should be understood that these Examples, while indicating preferred embodiments of the subject technology, are given by way of illustration only. From the above discussion and these Examples, one skilled in the art can ascertain the essential characteristics of the subject technology, and without departing from the spirit and scope thereof, an make various changes and modifications of the subject technology to adapt it to various uses and conditions.

### EXAMPLES

The subject technology is further defined in the following Examples. It should be understood that these Examples, while indicating preferred embodiments of the subject technology, are given by way of illustration only. From the above discussion and these Examples, one skilled in the art can ascertain the essential characteristics of the subject technology, and without departing from the spirit and scope thereof, can make various changes and modifications of the subject technology to adapt it to various uses and conditions.

### Example 1

A genomic and transcriptomic analysis was performed on Lasiodiplodia iraniensis, a species of jasmonic acid-producing fungus (Zheng et al, 2019), leading to the identification of a number of candidate sequences homologous to plant OPR genes (Schaller and Weiler, 1997). The candidate genes were then expressed in E. coli bacteria with an N-terminal His-tag and the product proteins were purified with an Ni-NTA purification system. An HPLC-based OPR activity assay was used to screen the candidate proteins, and the results showed that g5119 (SEQ ID NO:2) encodes a 12-oxophytodienoate reductase enzyme (SEQ ID NO: 1) in Lasiodiplodia iraniensis.

Codon-optimized g5119 (SEQ ID NO:3) was synthesized by Gene Universal Inc. (Newark, Delaware) and cloned into the pET-28a vector (Novagen, Wisconsin) between the NdeI and XhoI sites. The resulting construct was transformed into BL21(DE3) competent E. coli cells for expression. In a typical experiment, an overnight culture was used to inoculate liquid Luria-Bertani (LB) medium (2%) containing 100 mg/L of carbenicillin. The culture was first grown at 37° C. to are OD₆₀₀ of 0.6 and cooled down to 16° C. Then, 1 mM IPTG was added to induce protein expression. After 18 hours of incubation at 16° C. the cells were harvested by centrifugation and stored at −80° C. until use.

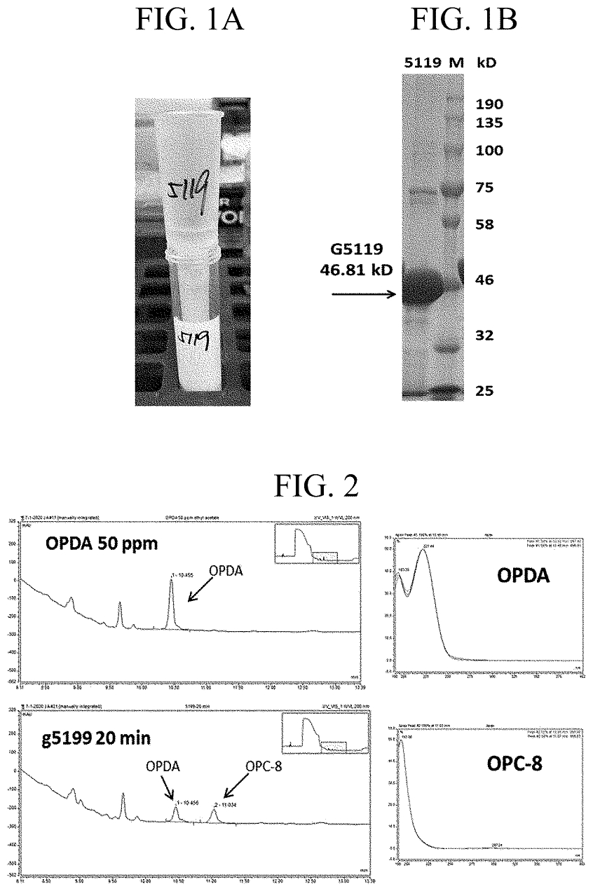

Total soluble proteins were extracted from frozen cells using B-PER(trademark) bacterial cell lysis reagents (Thermo Fisher Scientific, Massachusetts) according to the instructions from the manufacturer and further purified by Ni-NTA column (Qiagen, Maryland), as depicted in FIG. 1A. Purified g5119 protein was visualized on SDS-PAGE gel, as depicted in FIG. 1B.

The OPR activity assay was performed in 100 mM K-Pi buffer, pH 7.0 containing 50 mg/L OPDA (Cayman Chemical, Michigan), 1 mM NADPH and purified g5119 protein. The reaction was stopped by adding HCl to pH 2 and followed by ethyl acetate extraction. The ethyl acetate phase was used for HPLC analysis.

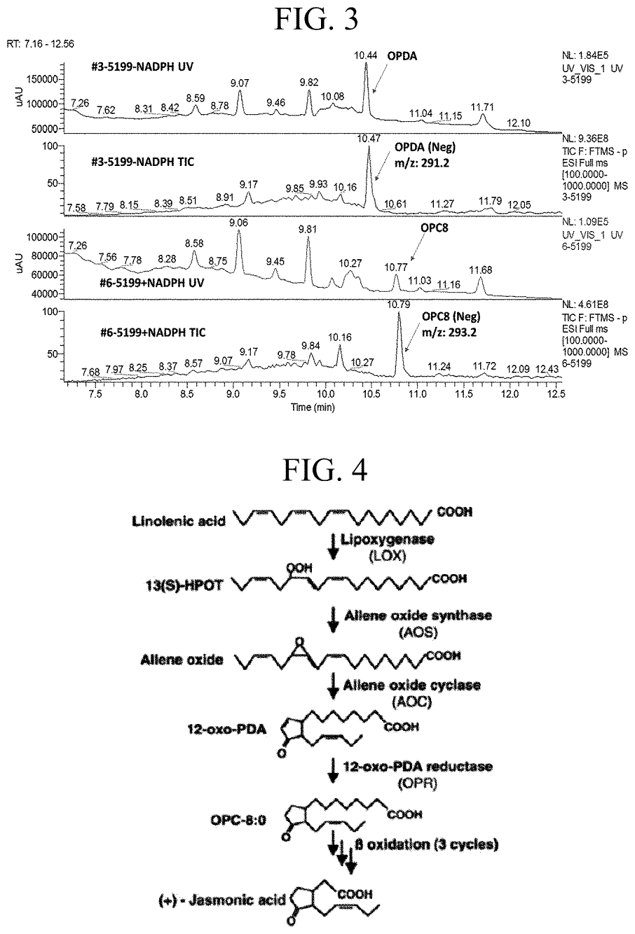

The HPLC analysis was performed on Thermo Scientific Vanquish UHPLC system using an Acclaim(trademark) 120, C18 column (3 μm 120 A, 3×150 mm). The mobile phases were: A, 0.1% TFA (trifluoroacetic acid) and B, acetonitrile with the gradient: 0-5 min, 5% B; 5-9 min, 5-80% B; 9-13 min, 80% B; 13-14 min, 80-5% B; 14-17 min, 5% B. The detector wavelength for OPDA and OPC8 was set at 200 nm. FIG. 2 includes a typical HPLC profile of a g5119-catalyzed reaction. Included in FIG. 3 are LC/MS analyses of g5119-catalyzed reactions. In #3-5199, the reaction was catalyzed by g5199 in the absence of NADPH; in #6-5199, the reaction was catalyzed by g5199 in the presence of 1 mM NADPH where no OPDA was detected after the reaction, indicating a complete conversion.

### Example 2

Agrobacterium tumefaciens-mediated transformation (ATMT) has been long used to transfer genes to a wide variety of fungi, including plant pathogenic or symbiotic fungi, (Vieira and Camilo, 2011). A. tumefaciens has the natural ability to transfer a segment of its Ti plasmid, known as ‘T-DNA’ to plant or fungal cells, becoming randomly integrated into nuclear chromosomes. The binary vector system may be used by ATMT. In this system, T-DNA and the virulence region are separated in 2 distinct plasmids, allowing easier genetic manipulation of the smaller binary vector containing the T-DNA (Hoekema et al., 1983) in mushrooms, industrial fungi, and biological control fungi (Ando et al., 2009; Sharma and Kuhad, 2010; Vieira and Camilo, 2011). A. tumefaciens has the natural ability to transfer a segment of its Ti plasmid, known as ‘T-DNA’ to plant or fungal cells, becoming randomly integrated into nuclear chromosomes. The binary vector system may be used by ATMT. In this system, T-DNA and the virulence region are separated in 2 distinct plasmids, allowing easier genetic manipulation of the smaller binary vector containing the T-DNA (Hoekema et al., 1983).

As reported in the literature, ATMT was successfully carried out in Lasiodiplodia theobromae (Muniz et al., 2014). An analogous ATMT-based transformation protocol can be used to achieve the expression and/or the overexpression of gs5119-like proteins in species of the Lasiodiplodia genus, for instance Lasiodiplodia iraniensis.

### Source and Growth Conditions of Microorganisms

The wild-type L. iraniensis is stored at 5° C. on potato dextrose agar (PDA) (Muniz et al., 2012). Escherichia coli strain DH5α is used as a host for the propagation of plasmid DNA. A. tumefaciens strain AGL1, which houses the binary vector, is maintained on Luria-Bertani (LB) medium supplemented with 250 μg/mL spectinomycin.

### Plasmid

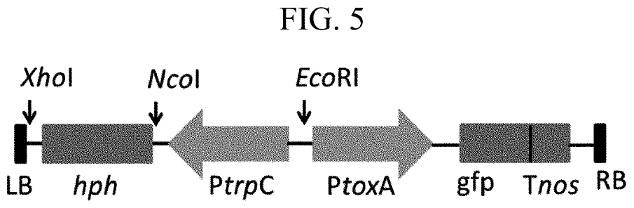

The backbone of pPm43GW (VB, Gent, Belgium) is used to construct the binary vector to transform L. iraniensis. It contains the cassette in which the E. coli hygromycin B (Hyg B) phosphotransferase (ph) resistance gene is under the regulation of the Aspergillus nidulans trpC promoter and the PtGFP cassette, which contains the promoter toxA-5′-UTR from Pyrenophora tritici-repentis, a gone coding for a polynucleotide sequence encoding a gs5119-like protein (“gs5119”), and the nos terminator (Tnos). The resulting plasmid is named as pPm43GW gs5119-HPH. As illustrated in FIG. 5, the gs5119 is placed under the control of the PtoxA-5′-UTR, and LB and RB represent left and right borders.

This vector is transformed into A. tumefaciens strain AGL1 using the electroporation method. The bacterium is spread onto an LB plate supplemented with 250 μg/mL spectinomycin, and incubated at room temperature for 2 days.

### Sensitivity to Hygromycin B Test

Before transformation, cultures obtained from germinated pycnidiospores (Muniz et al., 2012) are submitted to the minimum inhibitory concentration of hygromycin B. Cultures are inoculated on PDA, containing different concentrations of hygromycin B (0, 50, 100, 150, 200, 250, and 300 μg/mL) and, then, incubated at 28° C. for 14 days. For each treatment, 5 Petri dishes were used. Hygromycin B concentration that fully inhibits the mycelial growth of all 5 plates is used in the transformation experiment. This test is repeated twice.

### Fungal Transformation

Transformation is performed as previously described (de Groot et al., 1998; dos Reis et al., 2004; Staats et al., 2007), with minor modifications. Isolated colonies of A. tumefaciens, strain AGL1, are grown in 20 mL LB mannitol (10 g bacto-peptone, 5 g yeast extract, 2.5 g NaCl, 10 g mannitol) that are supplemented with 250 μg/n spectinomycin and 75 μg/1 mL carbenicillin overnight at 27° C., 150 rpm. Subsequently, A. tumefaciens cells are centrifuged and resuspend in 20 mL minimal medium (MM) (11.4 mM K₃HPO₄, 10.6 mM KH₂PO₄, 2.4 mM MgSO₄-7H₂O, 5.4 mM NaCl, 68 μM CaCl₂-2H₂O, 6.6 μM FeSO₄, 1.74 mM ZnSO₄-7H₂O, 2 mM CuSO₄-5H₂O, 8 mM H₃BO₃, 2.96 mM MnSO₄—H₂O, 2 mM Na₂MoO₄-2H₂O, 6.3 mM NH₄NO₃, 11 mM glucose) containing 75 μg/mL carbenicillin and 250 μg/mL spectinomycin. After incubation overnight at 27° C., 150 rpm, the culture is diluted to OD₆₀₀ of 0.15 in 20 mL induction medium (IM) (same as MM, but amended with 40 mM (2-[N-morpholino] ethane-sulfonic acid), 54 mM glycerol, and 200 μL acetosyringone (AS), containing 75 μg/mL carbenicillin and 250 μg/mL spectinomycin. The culture is returned to the same growth conditions until OD reaches 0.25. An L. iraniensis monosporic strain is grown on PDA at 28° C. for 14 days. Pycnidiospores are obtained according to the method of Muniz et al. (2012), are collected by flooding the Petri dish with sterile water, and counted under a microscope fitted with a Neubauer chamber. The final pycnidiospore concentration is adjusted to about 10⁷ spores/mL in saline solution. Co-cultivation between A. tumefaciens and pycnidiospores solution is performed by adding 100 μL bacterial culture to 100 μL fugal Suspension. This mixture is placed onto squared membranes of cellulose (120×120×17 mm) on a co-cultivation medium (IM+1.5% agar), in the presence and absence of 200 μL AS, and incubated for 2 days at 22° C. After 2 days, the samples are transferred to selection medium (SM) amended with 1.50 μg/mL hygromycin and 200 μM/mL, cefotaxime, to inhibit A. tumefaciens growth. The colonies that appear after incubation at 28° C. (putative transformants) are transferred to PDA with 150 μg/mL hygromycin and incubated at 28° C.

### Transgene Stability and Viability of Lasiodiplodia Transformants

All transgenic isolates from ATMT of Lasiodiplodia are subcultured on PDA amended with 150 μg hygromycin. The plates is incubated at 28° C. for 8 days. Every 2 days, the colony growth rate is checked. After 8 days, pycnidiospores production is tested. This procedure is successively repeated 3 times. Fungal colonics are then analyzed for GFP expression by fluorescence microscopy. The Hyg B minimum inhibitory concentration for L. iraniensis as determined by the cultivation of monosporic cultures on PDA containing different Hyg B concentrations. The minimum inhibitory concentration is then used for the initial selection of transformants.

### INDUSTRIAL APPLICABILITY

This disclosure has applicability in the nutraceutical and pharmacological industries. This disclosure relates generally to a method for the biosynthetic production of jasmonic acid, e.g., via a modified microbial strain.

SEQUENCES OF INTEREST

SEQ ID NO: 1
Amino acid sequence of wildtype 12-oxophytodienoate reductase enzyme gs5119 in
Lasiodiplodia iraniensis:
MSKPTQQLGQPLSFPISGRTAPNRFLKGAMSERLASWDPHPLSRGVPSRELAELYEVW
GRGQIGVLVTGNIMVDPAHPEAVGNAAIPFGASESSRRFEAFRDMARAGKKHGSLMI
AQLSHPGRQCDVRIQPDPVSASDVQLQGTVMGMRFGKPYAAGREEISRIVRQFQFAAT
YLEQAGFDGVEVHAAHGYLVAQFLSPTTNKRGDEYGGGSLEGRARLLVEIVRAIRAG
TSPGFVVGVKLNSVEFQQDGLRPEEARELCAVLDGMGVDFVELSGGTYEQLAFKHER
ESSRKREAFFIEFAALLAPELRRAKTYLTGGFQTAAGMADALATVDGVGLGRALCHEP
FLCRDILAGSTAGVARSLVDGNNAALSNAAALMQMWRLALGKEPWDTSTPEGMSA
MLQLLARYRKEFERADFTTPGDFRRTILAVL
SEQ ID NO: 2
DNA sequence of wild-type 12-oxophytodiencate reductase enzyme gs5119 in
Lasiodiplodia iraniensis:
ATGTCCAAACCCACTCAACAACTCGGCCAGCCCCTTTCATTTCCCATCTCCGGCAG
GACTGCCCCCAACCGGTTCCTCAAAGGAGCCATGTCGGAACGTCTTGCCTCCTGGG
ATCCGCATCCCCTCAGCCGCGGGGTGCCTTCGCGGGAGCTGGCGGAGCTTTACGA
GGTCTGGGGACGTGGCCAGATTGGCGTCCTCGTGACTGGAAACATCATGGTCGAC
CCCGCGCACCCGGAGGCCGTGGGCAACGCGGCTATCCCTTTTGGTGCGTCGGAAT
CATCCCGCCGGTTCGAAGCGTTCAGGGACATGGCACGCGCGGGAAAAAAGCACGG
AAGCCTGATGATTGCCCAACTATCTCATCCGGGCCGTCAGTGCGACGTGCGGATCC
AGCCTGACCCAGTGTCTGCCTCGGACGTCCAGCTCCAGGGGACAGTGATGGGGAT
GCGTTTCGGCAAGCCATACGCCGCAGGCCGCGAAGAAATTTCGAGGATCGTCCGA
CAGTTCCAATTCGCGGCCACGTACCTCGAGCAGGCTGGATTCGACGGCGTCGAGG
TGCACGCGGCCCACGGCTACCTCGTCGCGCAGTTCCTATCCCCGACGACCAACAA
GAGATCGTGCGTGCCATCCGCGCCGGCACCTCTCCCGGCTTCGTGGTCGGCGTCAA
GCTCAACAGCGTTGAGTTCCAGCAGGACGGGCTGCGGCCCGAGGAGGCGCGCGAG
CTGTGCGCCGTGCTGGACGGCATGGGCGTCGACTTTGTCGAGCTGTCCGGCGGCAC
CTACGAGCAGCTCGCCTTCAAACACGAGAGGGAATCCTCGAGGAAGCGCGAGGCC
TTCTTCATCGAGTTTGCTGCGCTGATTGCGCCGGAGTTGCGGAGGGCAAAGACGTA
CCTCACTGGCGGCTTCCAGACGGCTGCTGGCATGGCGGATGCGCTGGCGACTGTC
GATGGCGTGGGGCTCGGCCGCGCCCTGTGCCATGAGCCGTTTCTCTGTCGTGACAT
CTTGGCTGGGTCGACGGCTGGAGTGGCGAGGAGCCTCGTTGATGGGAATAATGCA
GCGTTGAGTAACGCCGCTGCCCTGATGCAAATGTGGAGACTCGCGCTGGGCAAGG
AGCCGTGGGATACCTCTACCCCGGAAGGTATGAGTGCTATGCTCCAGCTCCTTGCT
CGCTACCGGAAAGAGTTCGAGCGGGCAGATTTTACTACCCCCGGCGACTTTCGAA
GAACAATTCTTGCAGTGCTATAG
SEQ ID NO: 3
DNA sequence of wild-type 12-oxophytodienoate reductase enzyme gs5119 in
Lasiodiplodia iraniensis, codon optimized for Escherichia coli:
ATGAGTAAGCCGACCCAGCAGCTGGGTCAGCCGCTGAGCTTCCCGATTAGCGGTC
GTACCGCACCGAATCGCTTCCTGAAAGGTGCCATGAGCGAACGTCTGGCCAGCTG
GGATCCGCATCCGCTGAGCCGTGGTGTTCCGAGCCGTGAACTGGCCGAACTGTAT
GAAGTGTGGGGCCGTGGCCAGATTGGCGTTCTGGTGACCGGCAATATTATGGTTG
ATCCGGCCCATCCGGAAGCCGTTGGTAATGCCGCCATTCCGTTCGGTGCAAGTGAA
AGTAGCCGTCGCTTCGAAGCATTCCGTGATATGGCCCGCGCAGGCAAAAAACATG
CAGCCGGATCCGGTGAGTGCCAGCGATGTTCAGCTGCAGGGTACCGTGATGGGTA
TGCGCTTCGGTAAACCGTATGCCGCCGGTCGTGAAGAAATTAGCCGTATTGTTCGC
CAGTTCCAGTTCGCCGCCACCTATCTGGAACAGGCAGGCTTCGATGGTGTGGAAGT
GCATGCCGCCCATGGCTATCTGGTTGCCCAGTTCCTGAGTCCGACCACCAATAAAC
GTGGTGATGAATATGGTGGTGGTAGCCTGGAAGGTCGTGCCCGTCTGCTGGTTGA
AATTGTTCGTGCCATTCGTGCCGGCACCAGCCCGGGCTTCGTTGTGGGTGTGAAAC
TGAATAGTGTTGAATTCCAGCAGGATGGCCTGCGTCCGGAAGAAGCACGCGAACT
GTGTGCAGTTCTGGATGGTATGGGTGTTGACTTCGTGGAACTGAGTGGCGGTACCT
ATGAACAGCTGGCATTCAAACATGAACGCGAAAGCAGCCGTAAACGTGAAGCATT
CTTCATTGAATTCGCAGCCCTGATTGCACCGGAACTGCGTCGCGCCAAAACCTATC
TGACCGGCGGCTTCCAGACCGCCGCCGGTATGGCTGATGCACTGGCCACCGTTGAT
GGCGTTGGTCTGGGTCGCGCCCTGTGCCATGAACCGTTCCTGTGCCGCGATATTCT
GGCAGGCAGCACCGCAGGCGTTGCCGTAGTCTGGTGGATGGTAATAATGCCGCC
CTGAGCAATGCAGCAGCCCTGATGCAGATGTGGCGTCTGGCACTGGGTAAAGAAC
CGTGGGATACCAGTACCCCGGAAGGTATGAGCGCCATGCTGCAGCTGCTGGCCCG
TTATCGTAAAGAATTCGAACGCGCCGACTTCACCACCCCGGGTGACTTCCGTCGCA
CCATTCTGGCAGTTCTGTAA

1. A recombinant host cell comprising a metabolic pathway producing jasmonic acid, wherein the host cell overexpresses a polypeptide having 12-oxophytodienoate reductase (OPR) activity relative to a corresponding parental host cell, wherein the polypeptide comprises an amino acid sequence having at least 90% identity to the amino acid sequence as set forth in SEQ ID NO:1.

2. The recombinant host cell of claim 1, wherein the overexpressing the polypeptide having QPR activity has been provided by replacing a native promoter of a gene expressing the polypeptide having QPR activity with a promoter having a higher level of expression than the native promoter.

3. The recombinant host cell of claim 2, wherein the native promoter is replaced with the ToxA promoter of Pyrenophora tritici-repentis or the ToxB promoter of Pyrenophora tritici-repentis.

4. The recombinant host cell of claim 1, wherein the overexpressing of the polypeptide having OPR activity has been provided by recombinantly introducing into the host cell at least one copy of an exogenous polynucleotide sequence encoding the polypeptide having QPR activity.

5. The recombinant host cell of claim 1, wherein the host cell is selected from the group consisting of a bacterium, a yeast, a filamentous fungus, a cyanobacterial alga and a plant cell.

6. The recombinant host cell of claim 1, wherein the host cell is a filamentous fungus belonging to a genus selected from the group consisting of Lasiodiplodia, Rhizopus, Fusidium, Gibberella, Trichoderma, Hypocrea, Aspergillus, Fusarium, Penicillium, Neurospora, Chaetomium, Acremonium, Glomerella, Myceliophthora, Sporotrichum, Thielavia, Chrysosporium, Corynascus, Ctenomyces, Verticillium, Cordyceps, Nectria, and Magnaporthe.

7. The recombinant host cell of claim 6, wherein the host cell is a Lasiodiplodia iraniensis cell.

8. The recombinant host cell of claim 1, wherein the host cell is selected from the group consisting of Escherichia; Salmonella; Bacillus; Acinetobacter; Corynebacterium; Methylosinus; Methylomonas; Rhodococcus; Pseudomonas; Rhodobacter; Synechocystis; Aspergillus; Arthrobotrys; Brevibacteria; Microbacterium; Arthrobacter; Citrobacter; Klebsiella; Pantoea; Salmonella; Corynebacterium, and Clostridium.

9. The recombinant host cell of claim 1, wherein the host cell is a cell isolated from plants selected from the group consisting of soybean; rapeseed; sunflower; cotton; corn; tobacco; alfalfa; wheat; barley; oats; sorghum; rice; broccoli; cauliflower; cabbage; parsnips; melons; carrots; celery; parsley; tomatoes; potatoes; strawberries; peanuts; grapes; grass seed crops; sugar beets; sugar cane; beans; peas; rye; flax; hardwood trees; softwood trees; forage grasses; Arabidopsis thaliana; rice (Oryza sativa); Hordeum yulgaie; switchgrass (Panicum vigratum); Brachypodium spp.; Brassica spp.; and Crambe abyssinica.

10. A biosynthetic method for producing jasmonic acid, comprising: cultivating a recombinant host cell according to claim 1 in a culture medium; and recovering the jasmonic acid from at least one of the recombinant cell and the culture medium.

11. A recombinant host cell comprising a metabolic pathway producing jasmonic acid, wherein the host cell overexpresses a gene encoding for a polypeptide having 12-oxophytodienoate reductase (OPR) activity relative to a corresponding parental host cell, wherein the gene comprises a polynucleotide sequence having at least 90% identity to the polynucleotide sequence as set forth in SEQ ID NO:2.

12. The recombinant host cell of claim 11, wherein the overexpressing the gene encoding for a polypeptide having 12-oxophytodienoate reductase (OPR) activity has been provided by replacing a native promoter of a gene expressing the polypeptide having OPR activity with a promoter having a higher level of expression than the native promoter.

13. The recombinant host cell of claim 12, wherein the native promoter is replaced with the ToxA promoter of Pyrenophora tritici-repentis or the ToxB promoter of Pyrenophora tritici-repentis.

14. The recombinant host cell of claim 11, wherein the overexpressing the gene encoding for a polypeptide having 12-oxophytodienoate reductase (OPR) activity has been provided by recombinantly introducing into the host cell at least one copy of an exogenous sequence encoding the polypeptide having QPR activity.

15. The recombinant host cell of claim 11, wherein the host cell is selected from the group consisting of a bacterium, a yeast, a filamentous fungus, a cyanobacterial alga and a plant. cell.

16-19. (canceled)

20. A biosynthetic method for producing jasmonic acid, the method comprising:
cultivating the recombinant cell of claim 11 in a culture medium; and recovering the jasmonic acid from at least one of the recombinant cell and the culture medium.

21. A biosynthetic method for producing a polypeptide having 12-oxophytodienoate reductase (OPR) activity, the method comprising:
cultivating the recombinant cell of claim 11; and recovering the polypeptide having 12-oxophytodienoate reductase (OPR) activity from at least one of the recombinant cells and the culture medium.

22. A vector comprising a polynucleotide sequence having at least 90% identity to the polynucleotide sequence as set forth in SEQ ID NO:2.

23. (canceled)

24. A recombinant host cell overexpressing a gene encoding for a polypeptide having 12-oxophytodienoate reductase (OPR) activity relative to a corresponding parental host cell, wherein the gene comprises a polynucleotide sequence having at least 90% identity to the polynucleotide sequence as set forth in SEQ ID NO:3.

25. (canceled)

26. A biosynthetic method for producing jasmonic acid, comprising: cultivating a recombinant host cell according to claim 24; and recovering the jasmonic acid from at least one of the recombinant cell and the culture medium.
